(19) United States
(12) Patent Application Publication
(10) Pub. No.: US 2014/0322156 A1
(43) Pub. Date: Oct. 30, 2014

(54) PROCESS FOR REDUCING MALODOURS

(71) Applicant: Henkel AG & Co. KGaA, Duesseldorf (DE)

(72) Inventors: Ursula Huchel, Koeln (DE); Marc Weyhe, Krefeld (DE); Lukas Baron, Essen (DE); Frank Rittler, Duesseldorf (DE); Andreas Bauer, Kaarst (DE)

(21) Appl. No.: 14/327,782

(22) Filed: Jul. 10, 2014

Related U.S. Application Data
(63) Continuation of application No. PCT/EP2012/075719, filed on Dec. 17, 2012.

(30) Foreign Application Priority Data
Feb. 1, 2012 (DE) 10 2012 201 429.2

(51) Int. Cl. A61L 9/01; A01N 33/08; A61L 9/14

(52) U.S. Cl. CPC A61L 9/01; A61L 9/14; A01N 33/08
USPC 424/76.21; 424/76.1

(57) ABSTRACT

The use of 2-amino-1,3-propanediol and/or substituted 2-amino-1,3-propanediol for degradation of malodours caused by the presence of acids, thiols, sulphides, amines, aromatics, alcohols and/or heteroaromatics is described. By addition of 2-amino-1,3-propanediol and/or substituted 2-amino-1,3-propanediol to the ambient air or, for example, incorporated into washing compositions, it is possible to reduce corresponding malodours.

### FIELD OF THE INVENTION

The present invention generally relates to a method for degrading off-odors, preferably with regard to the treatment of hard and/or soft surfaces; it relates in particular to the degradation of off-odors in the context of a textile treatment method, and likewise to the degradation of off-odors in room air.

### BACKGROUND OF THE INVENTION

An important consumer requirement, which also plays a role e.g. in the utilization of washing, cleaning, or care-providing agents, consists in the elimination or at least reduction of bad odors (i.e. off-odors) or undesired odors. Off-odors derive from specific olfactorily active compounds that are also referred to as “malodorants.” Malodorants are foul-smelling compounds having so-called kakosmophoric groups, e.g. sulfur derivatives. The presence of such off-odors generally results in a negative effect on human comfort, and for that reason the consumer makes an effort to extinguish these odors. Often, however, the off-odors are not extinguished but merely masked. It is usual to use for this purpose products that contain volatile, usually pleasant-smelling substances, and that even in small quantities can mask foul odors.

In the existing art, international application WO 2010/142749 A1 describes the use of specific 2-amino-1,3-propanediols and/or substituted 2-amino-1,3-propanediols in order to degrade off-odors brought about the presence of aldehydes and/or ketones. The reduction of off-odors that are attributable to acids, thiols, sulfides, amines, aromatics, alcohols, and/or heteroaromatics is not discussed therein.

The object of the present invention was to provide the consumer with the ability to bring about a degradation of off-odors that are attributable to acids, thiols, sulfides, amines, aromatics, alcohols, and/or heteroaromatics.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method for degrading off-odors brought about by the presence of acids, thiols, sulfides, amines, aromatics, alcohols, and/or heteroaromatics, wherein 2-amino-1,3-propanediol and/or substituted 2-amino-1,3-propanediol in accordance with formula (I)

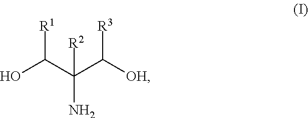

wherein in this formula the residues R¹, R², and R³, mutually independently in each case, denote hydrogen or hydrocarbon residues that can be acyclic or cyclic, substituted or unsubstituted, branched or unbranched, and saturated or unsaturated, is added.

A washing, cleaning, or care-providing method employing a washing, cleaning, or care-providing agent comprising 2-amino-1,3-propanediol and/or substituted 2-amino-1,3-propanediol in accordance with formula (I)

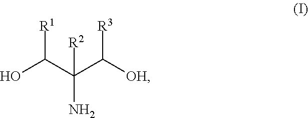

wherein in this formula the residues R¹, R², and R³, mutually independently in each case, denote hydrogen or hydrocarbon residues that can be acyclic or cyclic, substituted or unsubstituted, branched or unbranched, and saturated or unsaturated, preferably in quantities from 0.001 to 5 wt %, “wt %” based on the total agent, for degrading off-odors brought about by the presence of acids, thiols, sulfides, amines, aromatics, alcohols, and/or heteroaromatics.

Use of amino-1,3-propanediol and/or of substituted 2-amino-1,3-propanediol in accordance with formula (I) in preferably fragrance-containing washing, cleaning, or care-providing agents to degrade off-odors brought about by the presence of acids, thiols, sulfides, amines, aromatics, alcohols, and/or heteroaromatics.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The object stated above is achieved by the subject matter of the invention, namely a method for degrading off-odors brought about by the presence of acids, thiols, sulfides, amines, aromatics, alcohols, and/or heteroaromatics, wherein 2-amino-1,3-propanediol and/or substituted 2-amino-1,3-propanediol in accordance with formula (I)

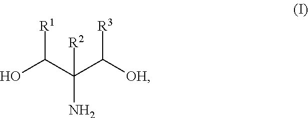

wherein in this formula the residues R¹, R², and R³, mutually independently in each case, denote hydrogen or hydrocarbon residues that can be acyclic or cyclic, substituted or unsubstituted, branched or unbranched, and saturated or unsaturated, is added. The hydrocarbon residues for purposes of the invention can in principle comprise heteroatoms, for example nitrogen, oxygen, or sulfur atoms. The hydrocarbon residues for purposes of the invention can in principle also be aromatic hydrocarbon residues.

R¹ and R³ in particular denote, mutually independently in each case, C₁₋₆ alkyl residues, preferably C₁₋₃ alkyl residues, or hydrogen. Particularly preferred residues R² are, besides hydrogen, in particular methyl, ethyl, and hydroxymethyl residues. In a preferred embodiment, R¹, R², and R³ each signify hydrogen.

2-Amino-2-(hydroxymethyl)propane-1,3-diol, 2-amino-1,3-propanediol(serinol), 2-amino-2-methylpropane-1,3-diol(methylserinol), and mixtures thereof are preferably usable for purposes of the invention.

The term “addition” is to be understood here in the broadest sense, i.e. 2-amino-1,3-propanediol and/or substituted 2-amino-1-3-propanediol is employed in order to diminish the aforesaid off-odors or undesired odors. Whenever an off-odor is present, there must be an object from which that off-odor is proceeding, or a space or system in which the off-odor is perceptible. According to the present invention, the 2-amino-1,3-propanediol and/or substituted 2-amino-1,3-propanediol can be delivered onto to that object or into that space or system.

The 2-amino-1,3-propanediol and/or substituted 2-amino-1,3-propanediol can, for example, be sprayed into the room air; it can be placed into a container with water or a washing bath; it can be placed directly onto any demarcated object, for example including a sweaty shirt, or a plurality of such objects, for example a washing load made up of many soiled shirts or the like.

It has been discovered, surprisingly, that the method according to the present invention makes possible an appreciable reduction, to the point of extinction, of bad odors or off-odors or undesired odors that are attributable to the presence of acids, thiols, sulfides, amines, aromatics, alcohols, and/or heteroaromatics.

Foul-smelling acids, thiols, sulfides, amines, aromatics, alcohols, and heteroaromatics are familiar to one skilled in the art; these are, for example, butyric acid, 1-butanethiol, dimethyl sulfide, 3-methyl-1H-indole, tetrahydrothiophene, etc.

The method according to the present invention is successfully usable in particular in order to minimize or extinguish off-odors with regard to foul-smelling textiles which result, for example, after sports activities, or with regard to off-odors in the toilet or WC sector.

In a preferred embodiment of the invention, the 2-amino-1,3-propanediol and/or substituted 2-amino-1,3-propanediol in accordance with formula (I) is used together with fragrances, in particular comprising fragrance aldehyde and/or fragrance ketone. Fragrance aldehydes are those fragrances which are chemically an aldehyde and which advantageously produce in humans an, in particular, pleasant olfactory sensation. Fragrance ketones are those fragrances which are chemically a ketone and which advantageously produce in humans a, in particular, pleasant olfactory sensation. Particularly suitable fragrance aldehydes and fragrance ketones are listed by way of example below.

The concentration of optional fragrances in the agent usable according to the present invention is preferably 0.001 to 10 wt %, advantageously 0.01 to 5 wt %, and in particular 0.1 to 3 wt %, “wt %” being based on the total agent. The combined use of 2-amino-1,3-propanediol and/or substituted 2-amino-1,3-propanediol with fragrances preferably comprising fragrance aldehydes and/or fragrance ketones, in particular in combination with surfactants, makes possible a very particularly good reduction of off-odors that is desirable according to the present invention.

According to a preferred embodiment of the invention, the fragrance aldehyde is selected from adoxal(2,6,10-trimethyl-9-undecenal), anisaldehyde(4-methoxybenzaldehyde), cymal(3-(4-isopropylphenyl)-2-methylpropanal), ethylvanillin, florhydral(3-(3-isopropylphenyl)butanal), helional(3-(3,4-methylenedioxyphenyl)-2-methylpropanal), heliotropin, hydroxycitronellal, lauraldehyde, lyral(3- and 4-(4-hydroxy-4-methylpentyl)-3-cyclohexene-1-carboxaldehyde), methyl nonyl acetaldehyde, lilial(3-(4-tert-butylphenyl)-2-methylpropanal), phenyl acetaldehyde, undecylenealdehyde, vanillin, 2,6,10-trimethyl-9-undecenal, 3-dodecen-1-al, alpha-n-amylcinnamaldehyde, melonal(2,6-dimethyl-5-heptenal), 2,4-dimethyl-3-cyclohexene-1-carboxaldehyde(triplal), 4-methoxybenzaldehyde, benzaldehyde, 3-(4-tert-butylphenyl)propanal, 2-methyl-3-(para-methoxyphenyl)propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha,alpha-dimethylhydrocinnamaldehyde, alpha-methyl-3,4-(methylenedioxy)hydrocinnamaldehyde, 3,4-methylenedioxybenzaldehyde, alpha-n-hexylcinnamaldehyde, m-cymene-7-carboxaldehyde, alpha-methyl phenyl acetaldehyde, 7-hydroxy-3,7-dimethyloctanal, undecenal, 2,4,6-trimethyl-3 -cyclohexene-1-carboxaldehyde, 4-(3)(4-methyl-3-pentenyl)-3-cyclohexenecarboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methane indane-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxaldehyde, 7-hydroxy-3,7-dimethyloctanal, trans-4-decenal, 2,6-nonadienal, para-tolylacetaldehyde, 4-methylphenyl acetaldehyde, 2-methyl-4-(2,6,6-trimethyl-1-cyclohexen-1-yl)-2-butenal, ortho-methoxycinnamaldehyde, 3,5,6-trimethyl-3-cyclohexenecarboxaldehyde, 3,7-dimethyl-2-methylene-6-octenal, phenoxyacetaldehyde, 5,9-dimethyl-4,8-decadienal, peony aldehyde(6,10-dimethyl-3-oxa-5,9-undecadien-1-al), hexahydro-4,7-methane indane-1-carboxaldehyde, 2-methyloctanal, alpha-methyl-4-(1-methyl ethyl)benzeneacetaldehyde, 6,6-dimethyl-2-norpinene-2-propionaldehyde, para-methyl phenoxyacetaldehyde, 2-methyl-3-phenyl-2-propen-1-al, 3,5,5-trimethylhexanal, hexahydro-8,8-dimethyl-2-naphthaldehyde, 3-propylbicyclo[2.2.1]-hept-5-ene-2-carbaldehyde, 9-decenal, 3-methyl-5-phenyl-1-pentanal, methyl nonyl acetaldehyde, hexanal, and trans-2-hexenal.

According to a preferred embodiment of the invention, the fragrance ketone is selected from methyl beta-naphthyl ketone, musk indanone(1,2,3,5,6,7-hexahydro-1,1,2,3,3-pentamethyl-4H-inden-4-one), tonalide(6-acetyl-1,1,2,4,4,7-hexamethyltetraline), alpha-damascone, beta-damascone, delta-damascone, isodamascone, damascenone, methyl dihydrojasmonate, menthone, carvone, camphor, koavone(3,4,5,6,6-pentamethylhept-3-en-2-one), fenchone, alpha-ionone, beta-ionone, gamma-methyl ionone, fleuramone(2-heptylcyclopentanone), dihydrojasmone, cis-jasmone, Iso-E-Super(1-(1,2,3,4,5,6,7,8-octahydro-2,3,8,8-tetramethyl-2-naphthalenyl)-ethan-1-one (and isomers)), methyl cedrenyl ketone, acetophenone, methyl acetophenone, para-methoxyacetophenone, methyl beta-naphthyl ketone, benzylacetone, benzophenone, para-hydroxyphenyl butanone, celery ketone(3-methyl-5-propyl-2-cyclohexenone), 6-isopropyldecahydro-2-naphthone, dimethyl octenone, Frescomenthe(2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, dulcinyl(4-(1,3-benzodioxol-5-yl)butan-2-one), hexalone(1-(2,6,6-trimethyl-2-cyclohexen-1-yl)-1,6-heptadien-3-one), isocyclemone E(2-acetonaphthone-1,2,3,4,5,6,7,8-octahydro-2,3,8,8-tetramethyl), methyl nonyl ketone, methyl cyclocitrone, methyl lavender ketone, orivone(4-tert-amyl cyclohexanone), 4-tert-butyl cyclohexanone, delphone(2-pentylcyclopentanone), muscone (CAS 541-91-3), neobutenone(1-(5,5-dimethyl-1-cyclohexenyepent-4-en-1-one), plicatone (CAS 41724-19-0), veloutone(2,2,5-trimethyl-5-pentylcyclopentan-1-one), 2,4,4,7-tetramethyloct-6-en-3-one, and tetrameran(6,10-dimethylundecen-2-one).

It is the case in principle that further optional fragrances that can be employed additionally in the method according to the present invention are subject to no particular restrictions, provided they contribute to the generation of an attractive scent note. Individual scent compounds of natural or synthetic origin, for example of the ester, ether, aldehyde, ketone, alcohol, and hydrocarbon types, can thus be used. Scent compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmecyclate. Ethers include, for example, benzyl ethyl ether and ambroxan; aldehydes, for example, the linear alkanals having 8 to 18 carbon atoms, citral, citronellal, citronellyloxyacetaldehyde, cyclamenaldehyde, 3-(4-propan-2-ylphenyl)butanal, lilial, and bourgeonal; ketones, for example, the ionones, α-isomethylionone and methyl cedryl ketone; alcohols include anethole, citronellol, eugenol, geraniol, linalool, phenylethyl alcohol, and terpineol; and hydrocarbons include principally terpenes such as limonene and pinene. Preferably, however, mixtures of different scents that together produce an attractive scent note are used.

It is also possible to employ natural fragrance mixtures such as those accessible from plant sources, for example pine, citrus, jasmine, patchouli, rose, or ylang-ylang oil. Also suitable are muscatel sage oil, chamomile oil, clove oil, lemon balm oil, mint oil, cinnamon leaf oil, linden blossom oil, juniper berry oil, vetiver oil, olibanum oil, galbanum oil, and labdanum oil, as well as orange blossom oil, neroli oil, orange peel oil, and sandalwood oil.

Further conventional fragrances that are usable in the context of the present invention are, for example, essential oils such as angelica oil, anise oil, arnica flower oil, basil oil, bay oil, champaca flower oil, silver fir oil, silver fir cone oil, elemi oil, eucalyptus oil, fennel oil, fir needle oil, galbanum oil, geranium oil, gingergrass oil, guaiac wood oil, balsam gurjun oil, helichrysum oil, ho oil, ginger oil, iris oil, cajeput oil, calamus oil, chamomile oil, camphor oil, kanaga oil, cardamom oil, cassia oil, pine needle oil, balsam copaiva oil, coriander oil, spearmint oil, caraway oil, cumin oil, lavender oil, lemon grass oil, lime oil, tangerine oil, lemon balm oil, ambrette seed oil, myrrh oil, clove oil, neroli oil, niaouli oil, olibanum oil, orange oil, oregano oil, palmarosa oil, patchouli oil, balsam peru oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, citron oil, and cypress oil, as well as ambrettolide, ambroxan, a-amylcinnamaldehyde, anethole, anisealdehyde, anise alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bomyl acetate, boisambrene forte, α-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvacrol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl β-naphthyl ketone, methyl n-nonyl acetaldehyde, methyl n-nonyl ketone, muscone, β-naphthol ethyl ether, β-naphthol methyl ether, nerol, n-nonylaldehyde, nonyl alcohol, n-octylaldehyde, p-oxyacetophenone, pentadecanolide, β-phenylethyl alcohol, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undelactone, vanillin, veratrumaldehyde, cinnamaldehyde, cinnamyl alcohol, cinnamic acid, cinnamic acid ethyl ester, cinnamic acid benzyl ester, diphenyl oxide, limonene, linalool, linalyl acetate and linalyl propionate, melusate, menthol, menthone, methyl-n-heptenone, pinene, phenylacetaldehyde, terpinyl acetate, citral, citronellal, and mixtures thereof.

According to a preferred embodiment of the invention, the method according to the present invention is directed toward the degradation of off-odors on hard and/or soft surfaces.

It is particularly preferable for the addition of 2-amino-1,3-propanediol and/or of substituted 2-amino-1,3-propanediol in accordance with formula (I) to occur by spraying. This corresponds to a preferred embodiment of the invention. If addition occurs by means of a spray can (pressurized-gas can, pressurized-gas package, aerosol package) or a pump atomizer to be operated mechanically (pump spray), forming a spray mist, a foam, a paste, or a liquid stream, a preferred embodiment of the invention once again exists.

According to a further preferred embodiment of the invention, the addition of 2-amino-1,3-propanediol and/or of substituted 2-amino-1,3-propanediol in accordance with formula (I) occurs in the context of a washing or cleaning process. Here the 2-amino-1,3-propanediol and/or substituted 2-amino-1,3-propanediol is employed in particular as a constituent of a preferably fragrance-containing washing, cleaning, or care-providing agent. Particularly good reduction of the bad odor occurs in this context.

A further subject of the invention is a washing, cleaning, or care-providing method employing a washing, cleaning, or care-providing agent comprising 2-amino-1,3-propanediol and/or substituted 2-amino-1,3-propanediol in accordance with formula (I)

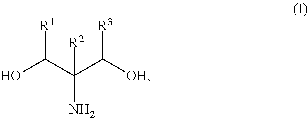

wherein in this formula the residues R¹, R², and R³, mutually independently in each case, denote hydrogen or hydrocarbon residues that can be acyclic or cyclic, substituted or unsubstituted, branched or unbranched, and saturated or unsaturated, preferably in quantities from 0.001 to 5 wt %, “wt %” based on the total agent, for degrading off-odors brought about by the presence of acids, thiols, sulfides, amines, aromatics, alcohols, and/or heteroaromatics.

The hydrocarbon residues recited can in principle comprise heteroatoms, for example nitrogen, oxygen, or sulfur atoms. The hydrocarbon residues recited can in principle also be aromatic hydrocarbon residues. R¹ and R³ in formula (I) in particular denote, mutually independently in each case, C₁₋₆ alkyl residues, preferably C₁₋₃ alkyl residues, or hydrogen. Particularly preferred residues R² are, besides hydrogen, in particular methyl, ethyl, and hydroxymethyl residues. In a preferred embodiment, R¹, R², and R³ each signify hydrogen.

2-Amino-2-(hydroxymethyl)propane-1,3-diol, 2-amino-1,3-propanediol(serinol), 2-amino-2-methylpropane-1,3-diol(methylserinol), and mixtures thereof are preferably usable for purposes of the invention.

It has been found, surprisingly, that a washing, cleaning, or care-providing method of this kind employing a corresponding washing, cleaning, or care-providing agent makes possible an appreciable reduction in bad odors or undesired odors that are attributable to the presence of acids, thiols, sulfides, amines, aromatics, alcohols, and/or heteroaromatics. In addition, it has been possible to achieve globally an improved scent experience, in particular in connection with textile treatment.

The washing, cleaning, or care-providing agent can in particular be a textile treatment agent in the form of a textile washing agent, fabric softener, softening washing agent, or washing adjuvant. It can also be, for example, a cleaning agent for hard surfaces, such as preferably a dishwashing agent, in particular an automatic dishwashing agent. It can also involve cleaning agents such as household cleaners, all-purpose cleaners, window cleaners, floor cleaners, etc. It can preferably be a product for cleaning toilet bowls and urinals, advantageously a flush cleaner for suspension in the toilet bowl or tank, in particular a so-called toilet block. In terms of the care-providing agent, this preferably refers to cosmetics, for example hair shampoos, deodorants, etc. that are usable for body care and/or body cleaning. Further care-providing agents can also be air-care products and room air fresheners.

According to a preferred embodiment, the agent usable according to the present invention is a textile treatment agent, an ironing adjuvant, a cleaning cloth, a textile washing agent, a fabric softener, a cleaning agent, in particular for hard and/or soft surfaces, a household cleaner, a care-providing agent, a washing care-providing agent, a room scenting agent, an air freshener, a conditioning agent, a coloring agent, a fabric softener, a conditioner substrate, a cleansing agent, a cosmetic agent, a bleaching agent, a deliming agent, an automobile care agent, floor care agent, oven care agent, leather care agent, furniture care agent, a scrubbing agent, a disinfectant, a scenting agent, a mold removal agent, and/or a precursor of the aforesaid agents. It is an advantage of the invention that the agents usable according to the present invention are highly shelf-stable.

The agents usable according to the present invention are in principle suitable equally for textile treatment and for cleaning hard surfaces, e.g. floors, and also for cosmetic use and for the air care sector or room air freshening or as an odor counteracting agent.

It is preferred in this context that the washing, cleaning, or care-providing agent that is used comprises 0.001 to 5 wt % fragrances, preferably comprising fragrance aldehydes and/or fragrance ketones.

It is furthermore preferred that the washing, cleaning, or care-providing agent that is used be present in solid form before utilization, preferably in powder form.

Suitable washing, cleaning, or care-providing agents in solid form, preferably in powder form or also in granulate form or in the form of pressed shaped elements, e.g. tablets, are provided in particular for use in aqueous systems, for example for use in a washing or cleaning bath.

The washing, cleaning, or care-providing agent that is used can, however, also be present in liquid form before utilization, preferably in gel form. This likewise corresponds to a preferred embodiment of the invention. Liquid agents can also be used directly, for example by application onto a foul-smelling object; optionally, they can be diluted beforehand. Utilization in aqueous systems is also possible.

If the washing, cleaning, or care-providing agent that is used contains at least 1 wt %, preferably at least 5 wt %, in particular at least 10 wt % surfactant, in particular anionic surfactant and/or nonionic surfactant, a further preferred embodiment of the invention then exists. A useful upper limit for surfactant can be, for example, 40 wt % or 30 wt % or 20 wt %, “wt %” being based in each case on the total agent. A particularly good reduction, desirable according to the present invention, in off-odors or undesired odors can thereby be effected.

The method according to the present invention is suitable for cleaning hard surfaces (e.g. dishes, floors, WCs) employing one of the washing, cleaning, or care-providing agents described previously.

If the method described above is a textile cleaning method or textile conditioning method in which the textile to be cleaned is subjected to textile laundering, preferably in an automatic washing machine, in particular at temperatures in the range from 15 to 60° C., even more advantageously 15 to 40° C., a preferred embodiment of the invention again exists.

A further subject of the invention is the use of amino-1,3-propanediol and/or substituted 2-amino-1,3-propanediol in accordance with formula (I) in (preferably fragrance-containing) washing, cleaning, or care-providing agents in order to degrade off-odors brought about by the presence of acids, thiols, sulfides, amines, aromatics, alcohols, and/or heteroaromatics.

According to a further preferred embodiment, the washing, care-providing, or cleaning agent usable according to the present invention comprises at least one, preferably multiple, active components, in particular components having washing, care-providing, or cleaning activity and/or cosmetic components, advantageously selected from the group comprising anionic surfactants, cationic surfactants, amphoteric surfactants, nonionic surfactants, acidifying agents, alkalizing agents, anti-creasing compounds, antibacterial substances, antioxidants, anti-redeposition agents, antistatic agents, builder substances, bleaching agents, bleach activators, bleach stabilizers, bleach catalysts, ironing adjuvants, co-builders, scents, shrinkage preventers, electrolytes, enzymes, color protectants, coloring agents, dyes, color transfer inhibitors, fluorescent agents, fungicides, germicides, odor-complexing substances, adjuvants, hydrotropes, rinse aids, complexing agents, preservatives, corrosion inhibitors, water-miscible organic solvents, optical brighteners, perfumes, perfume carriers, luster agents, pH adjusting agents, proofing and impregnation agents, polymers, swelling and anti-slip agents, foam inhibitors, sheet silicates, dirt-repelling substances, silver protectants, silicone oils, soil release active substances, UV protection substances, viscosity regulators, thickening agents, discoloration inhibitors, anti-gray agents, vitamins, and/or fabric softeners. For purposes of this invention, indications for the agent usable according to the present invention in wt % refer, unless otherwise indicated, to the total weight of the agent usable according to the present invention.

The quantities of the individual ingredients in the agents usable according to the present invention are aimed in each case toward the intended use of the relevant agent, and the skilled artisan is familiar in principle with the orders of magnitude of the quantities of ingredients to be used, or can gather them from the relevant technical literature. The surfactant content, for example, will be selected to be higher or lower depending on the intended use of the agents according to the present invention. For example, the surfactant content of, for example, washing agents can usually be equal to between 10 and 50 wt %, preferably between 12.5 and 30 wt %, and in particular between 15 and 25 wt %, while cleaning agents for automatic dishwashing can contain, for example, between 0.1 and 10 wt %, preferably between 0.5 and 7.5 wt %, and in particular between 1 and 5 wt % surfactants.

The agents usable according to the present invention can contain surfactants; anionic surfactants, nonionic surfactants, and mixtures thereof, but also cationic surfactants, are preferably appropriate. Suitable nonionic surfactants are in particular ethoxylation and/or propoxylation products of alkyl glycosides and/or of linear or branched alcohols each having 12 to 18 carbon atoms in the alkyl portion and 3 to 20, preferably 4 to 10, alkyl ether groups. Also usable are corresponding ethoxylation and/or propoxylation products of N-alkylamines, vicinal diols, fatty acid esters and fatty acid amides that correspond, in terms of the alkyl portion, to the aforesaid long-chain alcohol derivatives, and of alkylphenols having 5 to 12 carbon atoms in the alkyl residue.

Suitable anionic surfactants are, in particular, soaps, and those which contain sulfate or sulfonate groups having preferably alkali ions as cations. Usable soaps are preferably the alkali salts of saturated or unsaturated fatty acids having 12 to 18 carbon atoms. Such fatty acids can also be used in incompletely neutralized form. Included among the usable surfactants of the sulfate type are the salts of sulfuric acid semiesters of fatty alcohols having 12 to 18 carbon atoms, and sulfatization products of the aforesaid nonionic surfactants having a low degree of ethoxylation. Included among the usable surfactants of the sulfonate type are linear alkylbenzenesulfonates having 9 to 14 carbon atoms in the alkyl portion, alkanesulfonates having 12 to 18 carbon atoms, and olefinsulfonates having 12 to 18 carbon atoms that are produced upon reaction of corresponding monoolefins with sulfur trioxide, as well as alpha-sulfofatty acid esters that are produced upon sulfonation of fatty acid methyl or ethyl esters.

Cationic surfactants are preferably selected from esterquats and/or quaternary ammonium compounds (QACs) in accordance with the general formula (R^{I})(R^{II})(R^{III})(R^{IV})N⁺ X⁻, in which R^{I}to R^{IV} denote identical or different C₁₋₂₂ alkyl residues, C₇₋₂₈ alkyl residues, or heterocyclic residues, wherein two, or in the case of an aromatic bond such as in pyridine, even three residues form, together with the nitrogen atom, the heterocycle e.g. a pyridinium or imidazolinium compound, and X⁻ denotes halide ions, sulfate ions, hydroxide ions, or similar anions. QACs can be manufactured by the reaction of tertiary amines with alkylating agents such as methyl chloride, benzyl chloride, dimethyl sulfate, dodecyl bromide, but also ethylene oxide. The alkylation of tertiary amines having a long alkyl residue and two methyl groups can be achieved particularly easily, and the quaternization of tertiary amines having two long residues and one methyl group can also be carried out using methyl chloride under mild conditions. Amines that possess three long alkyl residues or hydroxy-substituted alkyl residues have low reactivity, and are quaternized, for example, using dimethyl sulfate. Suitable QACs are, for example, benzalkonium chloride(N-alkyl-N,N-dimethylbenzylammonium chloride), benzalkon B (m,p-dichlorobenzyldimethyl-C12 alkylammonium chloride), benzoxonium chloride (benzyldodecyl-bis(2-hydroxyethyl)ammonium chloride), cetrimonium bromide (N-hexadecyl-N,N-trimethylammonium bromide), benzetonium chloride (N,N-dimethyl-N-[2-[2-[p-(1,1,3,3-tetramethylbutyl)phenoxy]ethoxy]ethyl]benzylammonium chloride), dialkyldimethylammonium chlorides such as di-n-decyldimethylammonium chloride, didecyldimethylammonium bromide, dioctyldimethylammonium chloride, 1-cetylpyridinium chloride, and thiazoline iodide, as well as mixtures thereof. Preferred QACs are benzalkonium chlorides having C₈ to C22 alkyl residues, in particular C₁₂ to C₁₄ alkylbenzyldimethylammonium chloride.

“Esterquats” are to be understood here as preferably compounds of the general formula IV,

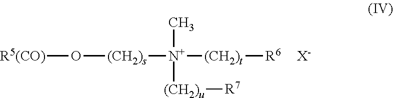

in which R⁵ denotes an alkyl residue or alkenyl residue having 12 to 22 carbon atoms and 0, 1, 2, or 3 double bonds; R⁶ and R⁷ mutually independently denote H, OH, or O(CO)R⁵; s, t, and u, mutually independently in each case, denote the value 1, 2, or 3; and X⁻ denotes an anion, in particular halide, methosulfate, methophosphate, or phosphate, as well as mixtures thereof. Compounds that contain the group O(CO)R⁵ for R⁶, and an alkyl residue having 16 to 18 carbon atoms for R⁵, are preferred. Compounds in which R⁷ additionally denotes OH are particularly preferred. Examples of compounds of formula (IV) are methyl-N-(2-hydroxyethyl)-N,N-di(tallowacyloxyethyl)ammonium metho sulfate, bis(palmitoyl)ethylhydroxyethylmethylammonium methosulfate, or methyl-N,N-bis(acyloxyethyl)-N-(2-hydroxyethyl)ammonium methosulfate. If quaternized compounds of formula (IV) that comprise unsaturated groups are used, those acyl groups whose corresponding fatty acids have an iodine number between 5 and 80, preferably between 10 and 60, and in particular between 15 and 45, and/or that have a cis/trans isomer ratio (in mol%) greater than 30:70, preferably greater than 50:50, and in particular greater than 70:30, are preferred. Commercially usual examples are the methylhydroxyalkyldialkoyloxyalkylammonium methosulfates marketed by the Stepan Company under the Stepantex® trademark, or the products of Cognis Deutschland GmbH known under the trade name Dehyquart® or the products of the manufacturer Goldschmidt-Witco known under the name Rewoquat®.

Surfactants are contained in the agents according to the present invention, if desired, in quantitative proportions preferably from 5 to 50 wt %, in particular from 8 to 30 wt %. In laundry post-treatment agents in particular, preferably up to 30 wt %, in particular 5 to 15 wt % surfactants are used, among them preferably cationic surfactants at least in part.

An agent usable according to the present invention preferably contains at least one water-soluble and/or water-insoluble, organic and/or inorganic builder. Included among the water-soluble organic builder substances are polycarboxylic acids, in particular citric acid and sugar acids, monomeric and polymeric aminopolycarboxylic acids, in particular methylglycinediacetic acid, nitrilotriacetic acid, and ethylenediaminetetraacetic acid, as well as polyaspartic acid, polyphosphonic acids, in particular aminotris(methylenephosphonic acid), ethylenediaminetetrakis(methylenephosphonic acid), and 1-hydroxyethane-1,1-diphosphonic acid, polymeric hydroxy compounds such as dextrin, as well as polymeric (poly)carboxylic acids, polymeric acrylic acids, methacrylic acids, maleic acids, and mixed polymers thereof, which can also contain, polymerized into them, small proportions of polymerizable substances having no carboxylic-acid functionality. The relative molecular weight of the homopolymers of unsaturated carboxylic acids is generally between 5000 and 200,000, that of the copolymers between 2000 and 200,000, preferably 50,000 to 120,000, based in each case on free acid. A particularly preferred acrylic acid-maleic acid copolymer has a relative molecular weight from 50,000 to 100,000. Suitable although less preferred compounds of this class are copolymers of acrylic acid or methacrylic acid with vinyl ethers, such as vinyl methyl ethers, vinyl esters, ethylene, propylene, and styrene, in which the proportion of acid is equal to at least 50 wt %. It is also possible to use, as water-soluble organic builder substances, terpolymers that contain two unsaturated acids and/or salts thereof as monomers and, as a third monomer, vinyl alcohol and/or a vinyl alcohol derivative or a carbohydrate. The first acidic monomer or salt thereof is derived from an ethylenically monounsaturated C₃ to C₈ carboxylic acid and preferably from a C₃ to C₄ monocarboxylic acid, in particular from (meth)acrylic acid. The second acidic monomer or salt thereof can be a derivative of a C₄ to C₈ dicarboxylic acid, maleic acid being particularly preferred. The third monomeric unit is constituted in this case by vinyl alcohol and/or preferably by an esterified vinyl alcohol. Vinyl alcohol derivatives that represent an ester of short-chain carboxylic acids, for example of C₁ to C₄ carboxylic acids, with vinyl alcohol, are particularly preferred. Preferred polymers contain 60 to 95 wt %, in particular 70 to 90 wt % (meth)acrylic acid or (meth)acrylate, particularly preferably acrylic acid or acrylate, and maleic acid or maleinate, as well as 5 to 40 wt %, preferably 10 to 30 wt % vinyl alcohol and/or vinyl acetate. Very particularly preferred in this context are polymers in which the weight ratio of (meth)acrylic acid or (meth)acrylate to maleic acid or maleinate is between 1:1 and 4:1, preferably between 2:1 and 3:1, and in particular 2:1 and 2.5:1. Both the quantities and the weight ratios are based on the acids. The second acidic monomer or salt thereof can also be a derivative of an allylsulfonic acid that is substituted in the 2-position with an alkyl residue, preferably with a C₁ to C₄ alkyl residue, or with an aromatic residue that is derived preferably from benzene or benzene derivatives. Preferred terpolymers contain 40 to 60 wt %, in particular 45 to 55 wt % (meth)acrylic acid or (meth)acrylate, particularly preferably acrylic acid or acrylate, 10 to 30 wt %, preferably 15 to 25 wt % methallylsulfonic acid or methallylsulfonate, and as a third monomer 15 to 40 wt %, preferably 20 to 40 wt % of a carbohydrate. This carbohydrate can be, for example, a mono-, di-, oligo-, or polysaccharide, mono-, di-, or oligosaccharides being preferred. Sucrose is particularly preferred. The use of the third monomer is presumed to incorporate defined break points into the polymer, which are responsible for the polymer's good biodegradability. These terpolymers generally have a relative molecular weight between 1000 and 200,000, preferably between 200 and 50,000, and in particular between 3000 and 10,000. Further preferred copolymers are those which comprise acrolein and acrylic acid/acrylic acid salts or vinyl acetate as monomers. Especially for the manufacture of liquid agents, the organic builder substance can be used in the form of aqueous solutions, preferably in the form of 30- to 50-weight-percent aqueous solutions. All the aforesaid acids are used as a rule in the form of water-soluble salts thereof, in particular alkali salts thereof.

Organic builder substances can be contained, if desired, in quantities of up to 40 wt %, in particular up to 25 wt %, and preferably from 1 to 8 wt %. Quantities close to the aforesaid upper limit are used preferably in pasty or liquid, in particular water-containing, agents usable according to the present invention. Laundry post-treatment agents usable according to the present invention, for example fabric softeners, according to the present invention can also optionally be free of organic builder.

Possibilities as water-soluble inorganic builder materials are, in particular, alkali silicates and polyphosphates, preferably sodium triphosphate. Crystalline or amorphous alkali aluminosilicates are used in particular as water-insoluble, water-dispersible inorganic builder materials, in quantities of up to 50 wt %, preferably not above 40 wt %, and in liquid agents in particular from 1 to 5 wt %. Among these, the crystalline sodium aluminosilicates of washing-agent quality, in particular zeolite A, P, and optionally X, are preferred. Quantities close to the aforesaid upper limit are preferably used in solid, particulate agents. Suitable aluminosilicates comprise, in particular, no particles having a particle size greater than 30 μm, and preferably are made up at a proportion of at least 80 wt % of particles having a size less than 10 μm.

Suitable substitutes or partial substitutes for the aforesaid aluminosilicate are crystalline alkali silicates, which can be present alone or mixed with amorphous silicates. The alkali silicates usable in the agents usable according to the present invention as builders preferably have a molar ratio of alkali oxide to SiO₂ below 0.95, in particular from 1:1.1 to 1:12, and can be present in amorphous or crystalline fashion. Preferred alkali silicates are sodium silicates, in particular amorphous sodium silicates, having a Na₂O:SiO₂ molar ratio from 1:2 to 1:2.8. Preferred crystalline silicates, which can be present alone or mixed with amorphous silicates, are crystalline sheet silicates of the general formula Na₂SiₓO₂ₓ₊₁.y H₂O in which x, the so-called “modulus,” is a number from 1.9 to 4 and y is a number from 0 to 20, and preferred values for x are 2, 3, or 4. Preferred crystalline sheet silicates are those in which x in the general formula recited assumes the values 2 or 3. In particular, both β- and δ-sodium disilicates (Na2Si₂O5.y H₂O) are preferred. Practically anhydrous crystalline alkali silicates manufactured from amorphous alkali silicates and having the aforesaid general formula, in which x denotes a number from 1.9 to 2.1, can also be used in agents usable according to the present invention. In a further preferred embodiment of agents usable according to the present invention, a crystalline sodium sheet silicate having a modulus from 2 to 3 is used, such as the one that can be manufactured from sand and soda. Crystalline sodium silicates having a modulus in the range from 1.9 to 3.5 are used in a further preferred embodiment of agents usable according to the present invention. The weight ratio of aluminosilicate to silicate, based in each case on anhydrous active substances, is preferably 1:10 to 10:1. In agents that contain both amorphous and crystalline alkali silicates, the weight ratio of amorphous alkali silicate to crystalline alkali silicate is preferably 1:2 to 2:1 and in particular 1:1 to 2:1.

Builder substances are contained in the agents usable according to the present invention preferably in quantities of up to 60 wt %, in particular from 5 to 40 wt %. Laundry post-treatment agents usable according to the present invention, for example fabric softeners, are preferably free of inorganic builders.

Peroxygen compounds that are suitable are, in particular, organic peracids or peracidic salts of organic acids such as phthalimidopercapronic acid, perbenzoic acid, or salts of diperdodecanedioic acid, hydrogen peroxide, and inorganic salts that release hydrogen peroxide under utilization conditions, such as perborate, percarbonate, and/or persilicate. If solid peroxygen compounds are to be used, they can be utilized in the form of powders or granulates, which in principle can also be encased in known fashion. Alkali percarbonate, alkali perborate monohydrate, or (in particular in liquid agents) hydrogen peroxide in the form of aqueous solutions that contain 3 to 10 wt % hydrogen peroxide, are used with particular preference. If an agent usable according to the present invention contains bleaching agents, such as preferably peroxygen compounds, the latter are present in quantities of preferably up to 50 wt %, in particular from 5 to 30 wt %. The addition of small quantities of known bleaching-agent stabilizers, for example phosphonates, borates or metaborates, and metasilicates, as well as magnesium salts such as magnesium sulfate, can be useful.

Compounds that, under perhydrolysis conditions, yield aliphatic peroxocarboxylic acids having preferably 1 to 10 carbon atoms, in particular 2 to 4 carbon atoms, and/or optionally substituted perbenzoic acid, can be used as bleach activators. Substances that carry the O- and/or N-acyl groups having the aforesaid number of carbon atoms, and/or optionally substituted benzoyl groups, are suitable. Multiply acylated alkylenediamines, in particular tetraacetylethylendiamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl or isononanoyl oxybenzenesulfonate (n- or iso-NOBS), carboxylic acid anhydrides, in particular phthalic acid anhydride, acylated polyvalent alcohols, in particular triacetin, ethylene glycol diacetate, 2,5-diacetoxy-2,5-dihydrofuran, and enol esters, as well as acetylated sorbitol and mannitol or mixtures thereof (SORMAN), acylated sugar derivatives, in particular pentaacetylglucose (PAG), pentaacetylfructose, tetraacetylxylose and octaacetyllactose, as well as acetylated, optionally N-alkylated glucamine and gluconolactone, and/or N-acylated lactams, for example N-benzoylcaprolactam, are preferred. Hydrophilically substituted acyl acetates and acyl lactams are likewise used in preferred fashion. Combinations of conventional bleach activators can also be used. Such bleach activators can be contained in the usual quantity range, preferably in quantities from 1 to 10 wt %, in particular 2 to 8 wt %, based on the total agent.

In addition to or instead of the conventional bleach activators presented above, sulfonimines and/or bleach-intensifying transition metal salts or transition metal complexes can also be contained as so-called bleach catalysts. Included among the appropriate transition metal compounds are, in particular, salen complexes of manganese, iron, cobalt, ruthenium, or molybdenum and nitrogen-analog compounds thereof, carbonyl complexes of manganese, iron, cobalt, ruthenium, or molybdenum, complexes of manganese, iron, cobalt, ruthenium, molybdenum, titanium, vanadium, and copper having nitrogen-containing tripod ligands, ammine complexes of cobalt, iron, copper, and ruthenium. Combinations of bleach activators and transition metal bleach catalysts can likewise be used. Bleach-intensifying transition metal complexes, in particular having the central atoms Mn, Fe, Co, Cu, Mo, V, Ti, and/or Ru, can be used in usual quantities, preferably in a quantity of up to 1 wt %, in particular from 0.0025 to 0.25 wt %, and particularly preferably from 0.01 to 0.1 wt %, based in each case on the entire agent.

Suitable enzymes usable in the agents are those from the class of proteases, cutinases, amylases, pullulanases, hemicellulases, cellulases, lipases, oxidases, and peroxidases, as well as mixtures thereof. Enzymatic active substances recovered from fungi or bacteria, such as Bacillus subtilis, Bacillus licheniformis, Streptomyces griseus, Humicola lanuginosa, Humicola insolens, Pseudomonas pseudoalcaligenes, or Pseudomonas cepacia, are particularly suitable. The enzymes that are optionally used can be adsorbed onto carrier substances and/or embedded into encasing substances in order to protect them from premature inactivation. They are contained, if desired, in the agents usable according to the present invention preferably in quantities not above 5 wt %, in particular from 0.2 to 2 wt %.

The agents can contain as optical brighteners, for example, derivatives of diaminostilbenedisulfonic acid or alkali metal salts thereof. Salts of 4,4′-bis(2-anilino-4-morpholino-1,3,5-triazinyl-6-amino)stilbene-2,2′-disulfonic acid, or compounds of similar structure that carry, instead of the morpholino group, a diethanolamino group, a methylamino group, an anilino group, or a 2-methoxyethylamino group, are suitable, for example. Brighteners of the substituted diphenylstyryl type can also be present, for example the alkali salts of 4,4′-bis(2-sulfostyryl)diphenyl, of 4,4′-bis(4-chloro-3-sulfostyryl)diphenyl, or of 4-(4-chlorostyryl)-4′-(2-sulfostyryl)diphenyl. Mixtures of the aforesaid brighteners can also be used.

Included among the suitable foam inhibitors are, for example, organopolysiloxanes and mixtures thereof with microfine, optionally silanated silicic acid, as well as paraffin waxes and mixtures thereof with silanated silicic acid or bis-fatty acid alkylenediamides. Mixtures of different foam inhibitors, for example those made of silicones, paraffins, or waxes, are also used with advantage. The foam inhibitors, in particular silicone- and/or paraffin-containing foam inhibitors, are preferably bound to a granular carrier substance that is soluble or dispersible in water. Mixtures of paraffins and bistearylethylenediamides are particularly preferred in this context.

In addition, the agents can also contain components that positively influence the ability of oils and fats to be washed out of textiles, so-called “soil release active agents”. This effect becomes particularly apparent when the soiled textile is one that has already been previously washed several times with an agent usable according to the present invention that contains this oil- and fat-releasing component. The preferred oil- and fat-releasing components include, for example, nonionic cellulose ethers such as methyl cellulose and methylhydroxypropyl cellulose having a 15 to 30 wt % proportion of methoxyl groups and a 1 to 15 wt % proportion of hydroxypropoxyl groups, based in each case on the nonionic cellulose ethers, as well as polymers, known from the existing art, of phthalic acid and/or of terephthalic acid or of derivatives thereof having monomeric and/or polymeric diols, in particular polymers of ethylene terephthalates and/or polyethylene glycol terephthalates or anionically and/or nonionically modified derivatives thereof.

The agents can also contain color transfer inhibitors, preferably in quantities from 0.1 to 2 wt %, in particular 0.1 to 1 wt %, which in a preferred embodiment of the invention are polymers of vinylpyrrolidone, vinylimidazole, vinylpyridine-N-oxide, or copolymers thereof. Also usable are both polyvinylpyrrolidones having molecular weights from 15,000 to 50,000 and polyvinylpyrrolidones having molecular weights above 1,000,000, in particular from 1,500,000 to 4,000,000, N-vinylimidazole/N-vinylpyrrolidone copolymers, polyvinyloxazolidones, copolymers based on vinyl monomers and carboxylic acid amides, pyrrolidone-group-containing polyesters and polyamides, grafted polyamidoamines and polyethylenimines, polymers having amide groups made up of secondary amines, polyamine-N-oxide polymers, polyvinyl alcohols, and copolymers based on acrylamidoalkenylsulfonic acids. It is also possible, however, to employ enzymatic systems comprising a peroxidase and hydrogen peroxide or a substance that yields hydrogen peroxide in water. The addition of a mediator compound for the peroxidase, for example an acetosyringone, a phenol derivative, or a phenothiazine or phenoxazine, is preferred in this case, wherein aforementioned conventional polymeric color-transfer-inhibiting active agents can also be additionally used. For use in agents usable according to the present invention, polyvinylpyrrolidone preferably has an average molar mass in the range from 10,000 to 60,000, in particular in the range from 25,000 to 50,000. Among the copolymers, those of vinylpyrrolidone and vinylimidazole at a molar ratio of 5:1 to 1:1, having an average molar mass in the range from 5000 to 50,000, in particular 10,000 to 20,000, are preferred.

The purpose of anti-gray agents is to keep dirt that has been detached from the textile fibers suspended in the bath. Water-soluble colloids, usually organic in nature, are suitable for this, for example starch, size, gelatin, salts of ether carboxylic acids or ether sulfonic acids of starch or of cellulose, or salts of acid sulfuric-acid esters of cellulose or of starch. Water-soluble polyamides containing acid groups are also suitable for this purpose. Starch derivatives other than those recited above can also be used, for example aldehyde starches. Cellulose ethers such as carboxymethyl cellulose (sodium salt), methyl cellulose, hydroxyalkyl cellulose, and mixed ethers such as methylhydroxyethyl cellulose, methylhydroxypropyl cellulose, methylcarboxymethyl cellulose, and mixtures thereof are preferably used, for example in quantities from 0.1 to 5 wt % based on the agent.

Included among the organic solvents that can be used in the agents usable according to the present invention, especially when the latter are present in liquid or pasty form, are alcohols having 1 to 4 carbon atoms, in particular methanol, ethanol, isopropanol, and tert-butanol, diols having 2 to 4 carbon atoms, in particular ethylene glycol and propylene glycol, as well as mixtures thereof, and the ethers derivable from the aforesaid compound classes. Water-miscible solvents of this kind are present in the agents usable according to the present invention, preferably in quantities not above 30 wt %, in particular from 6 to 20 wt %.

In order to establish a desired pH that does not result of itself from mixture of the other components, the agents usable according to the present invention can contain system-compatible and environmentally compatible acids, in particular citric acid, acetic acid, tartaric acid, malic acid, lactic acid, glycolic acid, succinic acid, glutaric acid, and/or adipic acid, but also mineral acids, in particular sulfuric acid, or bases, in particular ammonium hydroxides or alkali hydroxides. pH regulators of this kind are optionally contained in the agents usable according to the present invention in quantities preferably not above 20 wt %, in particular from 1.2 to 17 wt %.

The manufacture of solid agents usable according to the present invention presents no difficulties and can in principle occur in known fashion, for example by spray-drying or granulation; an optional peroxygen compound and optional bleach catalyst can, if applicable, be added later. A method comprising an extrusion step is preferred for the manufacture of agents usable according to the present invention having an elevated bulk weight, in particular in the range from 650 to 950 g/l. The manufacture of liquid agents usable according to the present invention likewise presents no difficulties and can likewise occur in known fashion.

According to a preferred embodiment, the teaching according to the present invention can be used to significantly reduce the perfume proportion in washing, cleaning, and toiletry agents. As a result, it is possible to offer perfumed products even for those particularly sensitive consumers who, because of specific incompatibilities and irritations, can use normally perfumed products only to a limited extent or not at all. Mention may be made in this connection chiefly of skin care products and deodorants, but also of washing agents such as, for example, hand washing agents.

A preferred solid, in particular powdered, washing agent usable according to the present invention can also in particular contain, besides the constituents to be employed according to the present invention (i.e. 2-amino-1,3-propanediol and/or substituted 2-amino-1,3-propanediol), components that are selected, for example, from the following:
anionic surfactants such as preferably alkylbenzenesulfonate, alkyl sulfate, e.g. in quantities preferably from 5 to 30 wt %,
nonionic surfactants such as preferably fatty alcohol polyglycol ethers, alkyl polyglucoside, fatty acid glucamide, e.g. in quantities preferably from 0.5 to 15 wt %,
builders, for example zeolite, polycarboxylate, sodium citrate, in quantities from, for example, 0 to 70 wt %, advantageously 5 to 60 wt %, preferably 10 to 55 wt %, in particular 15 to 40 wt %,
alkalis, for example sodium carbonate, in quantities e.g. from 0 to 35 wt %, advantageously 1 to 30 wt %, preferably 2 to 25 wt %, in particular 5 to 20 wt %,
bleaching agents, for example sodium perborate, sodium percarbonate, in quantities e.g. from 0 to 30 wt %, advantageously 5 to 25 wt %, preferably 10 to 20 wt %,
corrosion inhibitors, e.g. sodium silicate, in quantities e.g. from 0 to 10 wt %, advantageously 1 to 6 wt %, preferably 2 to 5 wt %, in particular 3 to 4 wt %,
stabilizers, e.g. phosphonates, advantageously 0 to 1 wt %,
foam inhibitor, e.g. soap, silicone oils, paraffins, advantageously 0 to 4 wt %, preferably 0.1 to 3 wt %, in particular 0.2 to 1 wt %,
enzymes, e.g. proteases, amylases, cellulases, lipases, advantageously 0 to 2 wt %, preferably 0.2 to 1 wt %, in particular 0.3 to 0.8 wt %,
anti-gray agent, e.g. carboxymethyl cellulose, advantageously 0 to 1 wt %,
discoloration inhibitor, e.g. polyvinylpyrrolidone derivatives, e.g. 0 to 2 wt %,
adjusting agent, e.g. sodium sulfate, advantageously 0 to 20 wt %,
optical brightener, e.g. stilbene derivative, biphenyl derivative, advantageously 0 to 0.4 wt %, in particular 0.1 to 0.3 wt %,
scents, preferably comprising fragrance aldehydes and/or fragrance ketones,
optionally water,
optionally soap,
optionally bleach activators,
optionally cellulose derivatives,
optionally dirt repellents,
   “wt %” being based in each case on the total agent.

In another preferred embodiment of the invention, the washing, cleaning, or care-providing agent is present in liquid form, preferably in gel form. Preferred liquid washing, cleaning, or care-providing agents have water contents of, for example, 10 to 95 wt %, preferably 20 to 80 wt %, and in particular 30 to 70 wt %, based on the total agent. In the case of liquid concentrates the water content can also be particularly low, e.g. <30 wt %, preferably <20 wt %, in particular <15 wt %, “wt %” being based in each case on the total agent. The liquid consumer products can also contain non-aqueous solvents.

A preferred liquid, in particular gel-type, washing agent usable according to the present invention can in particular also contain, besides the constituents to be employed according to the present invention (i.e. 2-amino-1,3-propanediol and/or substituted 2-amino-1,3-propanediol), components that are selected e.g. from the following:
anionic surfactants such as preferably alkylbenzenesulfonate, alkyl sulfate, e.g. in quantities preferably from 5 to 40 wt %,
nonionic surfactants such as preferably fatty alcohol polyglycol ethers, alkyl polyglucoside, fatty acid glucamide, e.g. in quantities preferably from 0.5 to 25 wt %,
builders, for example zeolite, polycarboxylate, sodium citrate, advantageously 0 to 15 wt %, preferably 0.01 to 10 wt %, in particular 0.1 to 5 wt %,
foam inhibitor, e.g. soap, silicone oils, paraffins, in quantities e.g. from 0 to 10 wt %, advantageously 0.1 to 4 wt %, preferably 0.2 to 2 wt %, in particular 1 to 3 wt %,
enzymes, e.g. proteases, amylases, cellulases, lipases, in quantities e.g. from 0 to 3 wt %, advantageously 0.1 to 2 wt %, preferably 0.2 to 1 wt %, in particular 0.3 to 0.8 wt %,
optical brightener, e.g. stilbene derivative, biphenyl derivative, in quantities e.g. from 0 to 1 wt %, advantageously 0.1 to 0.3 wt %, in particular 0.1 to 0.4 wt %,
scents, preferably comprising fragrance aldehydes and/or fragrance ketones,
optionally stabilizers,
water,
optionally soap, in quantities e.g. from 0 to 25 wt %, advantageously 1 to 20 wt %, preferably 2 to 15 wt %, in particular 5 to 10 wt %,
optionally solvents (preferably alcohols), advantageously 0 to 25 wt %, preferably 1 to 20 wt %, in particular 2 to 15 wt %,
   “wt %” being based in each case on the total agent.

A preferred liquid fabric softener usable according to the present invention can in particular also contain, besides the constituents to be employed according to the present invention (i.e. 2-amino-1,3-propanediol and/or substituted 2-amino-1,3-propanediol), components that are selected from the following:
cationic surfactants, such as especially esterquats, e.g. in quantities from 5 to 30 wt %,
co-surfactants, for example glycerol monostearate, stearic acid, fatty alcohols, fatty alcohol ethoxylates, e.g. in quantities from 0 to 5 wt %, preferably 0.1 to 4 wt %,
emulsifier agents, for example fatty amine ethoxylates, e.g. in quantities from 0 to 4 wt %, preferably 0.1 to 3 wt %,
scents, preferably comprising fragrance aldehydes and/or fragrance ketones,
dyes, preferably in the ppm range,
stabilizers, preferably in the ppm range,
solvents such as, in particular, water, in quantities preferably from 60 to 90 wt %,
   “wt %” being based in each case on the total agent.

### EXAMPLES

An experimental container having a capacity of 35 liters was equipped with a 1-cm opening for attachment of a hose with which an olfactory check of the atmosphere present therein could take place. Using a lateral hatch near the bottom, the container could be quickly opened and populated with investigative media (see below).

The experiment took place at room temperature under standard pressure. Two identically configured containers were used. A Petri dish having 5 ml of a 10-percent off-odor solution A (in polyethylene glycol 2000) was placed into the cleaned and odor-neutral containers. This off-odor solution A (see formula below) serves to simulate an off-odor that is usual for toilet areas. After one hour, the Petri dish was removed. After 30 minutes, an olfactory check of each the two containers yielded an intense foul toilet off-odor. A test solution (see below) was introduced in finely distributed form into one of the experimental containers by means of a spray bottle. One spray burst having a volume of 0.1 ml was generated in each case. The solvent used was water. The solutions had a 10-percent active agent content. Immediately after the spray burst and at the time intervals indicated in the table, an assessment of the odor intensity in the chamber was carried out. The scale of odor intensity extends from strong (score 10) to imperceptible (score 0). The assessment was performed by people with olfactory training.

Results

| | Intensity | Intensity | Intensity | Intensity | Intensity |
|---|---|---|---|---|---|
| | after 0 hr | after 1 hr | after 2 hr | after 4 hr | after 8 hr |
| Chamber | 10 | 3 | 1 | 1 | 0 |
| treated | | | | | |
| with serinol | | | | | |
| solution | | | | | |
| Chamber not | 10 | 6 | 6 | 5 | 4 |
| treated with | | | | | |
| a serinol | | | | | |
| solution | | | | | |
| Chamber | 10 | 4 | 2 | 1 | 0 |
| treated with | | | | | |
| methyl- | | | | | |
| serinol | | | | | |
| solution | | | | | |

The addition of serinol or methylserinol to the system therefore made possible a degradation of the off-odor.

Formula of the off-odor solution A used (toilet odor):

| | |
|---|---|
| 3-Methylbutanoic acid | 20 wt % |
| n-Butanoic acid | 20 wt % |
| n-Hexanoic acid | 20 wt % |
| 2,3-benzopyrrole | 20 wt % |
| 4-methyl-2,3-benzopyrrole | 10 wt % |
| Ammonia solution (25% in water) | 10 wt % |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for degrading off-odors brought about by the presence of acids, thiols, sulfides, amines, aromatics, alcohols, and/or heteroaromatics on hard and/or soft surfaces or room air, wherein 2-amino-1,3-propanediol and/or substituted 2-amino-1,3-propanediol in accordance with formula (I) 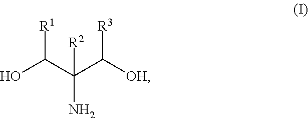
where in this formula the residues R¹, R², and R³, mutually independently in each case, denote hydrogen or hydrocarbon residues that can be acyclic or cyclic, substituted or unsubstituted, branched or unbranched, and saturated or unsaturated, is delivered onto the hard and/or soft surface or into the room air.

2. The method in accordance with claim 1, wherein 2-amino-1,3-propanediol and/or substituted 2-amino-1,3-propanediol in accordance with formula (I) is used together with fragrances comprising:
(a) a fragrance aldehyde selected from adoxal(2,6,10-trimethyl-9-undecenal), anisaldehyde(4-methoxybenzaldehyde), cymal(3-(4-isopropylphenyl)-2-methylpropanal), ethylvanillin, florhydral(3-(3-isopropylphenyl)butanal), helional(3-(3,4-methylenedioxyphenyl)-2-methylpropanal), heliotropin, hydroxycitronellal, lauraldehyde, lyral(3- and 4-(4-hydroxy-4-methylpentyl)-3-cyclohexene-1-carboxaldehyde), methyl nonyl acetaldehyde, lilial(3-(4-tert-butylphenyl)-2-methylpropanal), phenyl acetaldehyde, undecylenealdehyde, vanillin, 2,6,10-trimethyl-9-undecenal, 3-dodecen-1-al, alpha-n-amylcinnamaldehyde, melonal(2,6-dimethyl-5-heptenal), 2,4-dimethyl-3-cyclohexene-1-carboxaldehyde(triplal), 4-methoxybenzaldehyde, benzaldehyde, 3-(4-tert-butylphenyl)propanal, 2-methyl-3-(para-methoxyphenyl)propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 1,2,3,4,5,6,7,8-octahydro-8,8-dimethyl-2-naphthaldehyde, 2,4-dimethyl-3 -cyclohexene-1 -carboxaldehyde, 2-methyl-3-(isopropylphenyl)propanal, 1 -decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha,alpha-dimethylhydrocinnamaldehyde, alpha-methyl-3,4-(methylenedioxy)hydrocinnamaldehyde, 3,4-methylenedioxybenzaldehyde, alpha-n-hexylcinnamaldehyde, m-cymene-7-carboxaldehyde, alpha-methyl phenyl acetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methane indane-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxaldehyde, 7-hydroxy-3,7-dimethyloctanal, trans-4-decenal, 2,6-nonadienal, para-tolylacetaldehyde, 4-methylphenyl acetaldehyde, 2-methyl-4-(2,6,6-trimethyl-1-cyclohexen-1-yl)-2-butenal, ortho-methoxycinnamaldehyde, 3,5,6-trimethyl-3-cyclohexenecarboxaldehyde, 3,7-dimethyl-2-methylene-6-octenal, phenoxyacetaldehyde, 5,9-dimethyl-4,8-decadienal, peony aldehyde(6,10-dimethyl-3-oxa-5,9-undecadien-1-al), hexahydro-4,7-methane indane-1-carboxaldehyde, 2-methyloctanal, alpha-methyl-4-(1-methyl ethyl)benzeneacetaldehyde, 6,6-dimethyl-2-norpinene-2-propionaldehyde, para-methyl phenoxy acetaldehyde, 2-methyl-3-phenyl-2-propen-1-al, 3,5,5-trimethylhexanal, hexahydro-8,8-dimethyl-2-naphthaldehyde, 3-propylbicyclo[2.2.1]-hept-5-ene-2-carbaldehyde, 9-decenal, 3-methyl-5-phenyl-l-pentanal, methyl nonyl acetaldehyde, hexanal, and trans-2-hexenal,
and/or
(b) a fragrance ketone selected from methyl beta-naphthyl ketone, musk indanone(1,2,3,5,6,7-hexahydro-1,1,2,3,3-pentamethyl-4H-inden-4-one), tonalide(6-acetyl-1,1,2,4,4,7-hexamethyltetraline), alpha-damascone, beta-damascone, delta-damascone, isodamascone, damascenone, methyl dihydrojasmonate, menthone, carvone, camphor, koavone(3,4,5,6,6-pentamethylhept-3-en-2-one), fenchone, alpha-ionone, beta-ionone, gamma-methyl ionone, fleuramone(2-heptylcyclopentanone), dihydrojasmone, cis-jasmone, Iso-E-Super (1-(1,2,3,4,5,6,7,8-octahydro-2,3,8,8-tetramethyl-2-naphthalenyl)-ethan-1-one (and isomers)), methyl cedrenyl ketone, acetophenone, methyl acetophenone, para-methoxyacetophenone, methyl beta-naphthyl ketone, benzylacetone, benzophenone, para-hydroxyphenyl butanone, celery ketone(3-methyl-5-propyl-2-cyclohexenone), 6-isopropyldecahydro-2-naphthone, dimethyl octenone, Frescomenthe(2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1 -yl)propyl)cyclopentanone, 1-(p-menthen-6(2)yl)-1-propanone, 4-(4-hydroxy-3-methoxyphenyl)-2-butanone, 2-acetyl-3,3-dimethylnorbomane, 6,7-dihydro-1,1,2,3,3-pentamethyl-4(5H)-indanone, 4-damascol, dulcinyl(4-(1,3-benzodioxol-5-yl)butan-2-one), hexalone(1-(2,6,6-trimethyl-2-cyclohexen-1-yl)-1,6-heptadien-3-one), isocyclemone E (2-acetonaphthone-1,2,3,4,5,6,7,8-octahydro-2,3,8,8-tetramethyl), methyl nonyl ketone, methyl cyclocitrone, methyl lavender ketone, orivone(4-tert-amyl cyclohexanone), 4-tert-butyl cyclohexanone, delphone(2-pentylcyclopentanone), muscone (CAS 541-91-3), neobutenone(1-(5,5-dimethyl-1-cyclohexenyl)pent-4-en-1-one), plicatone (CAS 41724-19-0), veloutone(2,2,5-trimethyl-5-pentylcyclopentan-1-one), 2,4,4,7-tetramethyloct-6-en-3-one, and tetrameran (6, 10-dimethylundecen-2-one).

3. The method in accordance with claim 1, wherein the delivery of 2-amino-1,3-propanediol and/or of substituted 2-amino-1,3-propanediol in accordance with formula (I) occurs by spraying.

4. The method in accordance with claim 3, wherein that addition occurs by means of a spray can (pressurized-gas can, pressurized-gas package, aerosol package) or a pump atomizer to be operated mechanically (pump spray), forming a spray mist, a foam, a paste, or a liquid stream.

5. The method in accordance with claim 1, wherein the delivery of 2-amino-1,3-propanediol and/or of substituted 2-amino-1,3-propanediol in accordance with formula (I) occurs in the context of a washing or cleaning process.

6. The method according to claim 1, wherein 2-amino-1,3-propanediol and/or substituted 2-amino-1,3-propanediol in accordance with formula (I) is delivered as a constituent of a washing, cleaning, or care-providing agent.

7. A washing, cleaning, or care-providing method for degrading off-odors brought about by the presence of acids, thiols, sulfides, amines, aromatics, alcohols, and/or heteroaromatics on hard or soft surfaces wherein a washing, cleaning, or care-providing agent comprising from 0.001 to 5 wt %, “wt %” based on the total agent 2-amino-1,3-propanediol and/or substituted 2-amino-1,3-propanediol in accordance with formula (I) 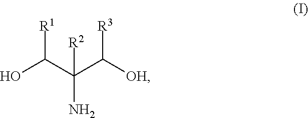
wherein in this formula the residues R¹, R², and R³, mutually independently in each case, denote hydrogen or hydrocarbon residues that can be acyclic or cyclic, substituted or unsubstituted, branched or unbranched, and saturated or unsaturated, is delivered onto the hard or soft surface.

8. The method according to claim 7, wherein the washing, cleaning, or care-providing agent employed comprises 0.001 to 5 wt % fragrances.

9. The method according to claim 7, wherein the washing, cleaning, or care-providing agent employed is present in solid form before utilization.

10. The method according claim 7, wherein the washing, cleaning, or care-providing agent employed is present in liquid form before utilization

11. The method according to claim 8, wherein the washing, cleaning, or care-providing agent employed contains at least 1 wt % anionic surfactant and/or nonionic surfactant.

12. The method according to claim 8, wherein it is a textile cleaning method or textile conditioning method in which the textile to be cleaned is subjected to a textile laundering in an automatic washing machine at temperatures in the range from 15 to 60° C.
